(19) United States
(12) Patent Application Publication
(10) Pub. No.: US 2023/0115063 A1
(43) Pub. Date: Apr. 13, 2023

(54) BIOFILM ENHANCED TEXTILE AND METHODS FOR MANUFACTURING THEREOF

(71) Applicant: LULULEMON ATHLETICA CANADA INC., Vancouver (CA)

(72) Inventors: Lynn Yuqin Wan, Vancouver (CA); Robert John Gathercole, Vancouver (CA); Felix Moser, Vancouver (CA); Noemie-Manuelle DORVAL COURCHESNE, Montreal (CA); Dalia Jane Saldanha, Montreal (CA); Zahra Adbali, Montreal (CA); Anqi Cai, Montreal (CA); Masoud Aminzare, Montreal (CA)

(21) Appl. No.: 17/913,678

(22) PCT Filed: Mar. 31, 2021
(86) PCT No.: PCT/CA2021/050437

§ 371 Date: Sep. 22, 2022

Related U.S. Application Data
(60) Provisional application No. 63/003,455, filed on Apr. 1, 2020.

(51) Int. Cl. D06M 16/00; B82Y 5/00; A61K 35/74

(52) U.S. Cl. CPC D06M 16/003; B82Y 5/00; A61K 35/74; D06M 2101/02

(57) ABSTRACT

Biofilm-enhanced textiles and methods of preparing same are disclosed. The biofilm-enhanced textiles include a textile; and a biofilm comprising one or more microorganisms and an extracellular material, the one or more microorganisms being engineered to express one or more proteins on a cell surface to adhere to the textile, and to express one or more genes that produce a response to changes within the textile or to an environment surrounding the textile.

### TECHNICAL FIELD

The present disclosure relates to the field of wearable textiles, and more specifically, to a wearable textile containing living microorganisms or microbes and methods for manufacturing such textiles.

### BACKGROUND

Textile performance is traditionally considered in terms of a textile's properties associated with its mechanical performance such as, for example, stretchability, bendability, thermal insulation, water resistance, breathability, moisture wicking, durability, hand feel, fit, texture and physical and chemical stability. While these properties are valuable, modern consumers' expectations have grown to include improved performance, diverse capabilities and low environmental footprint. For example, it is desirable that new fabrics have more advanced properties, such as, for example, include electronic wearables that record, report, and respond to physiological signals such as heart rate, sweat production, dissolved blood oxygen, excess strain, unbalanced movement, step count, stress, etc.

Living microbes often form mechanically and chemically robust composites of cells and extracellular material. These composites, termed biofilms, have been shown to contain living cells that continue to sense and respond to their environment. Biofilms are complex composites of one or more types of cells and extracellular matrix components that anchor the cells in place. Biofilms can be genetically modified to exhibit a wide variety of desirable attributes.

Thus, there remains a need for textiles that are more responsive to their immediate environment.

### SUMMARY

In one aspect, a biofilm-enhanced textile is provided. The biofilm-enhanced textile comprises a textile and a biofilm that includes one or more microorganisms and an extracellular material. The microorganisms are engineered to express proteins on their surface to adhere to the textile. The biofilm acts as a biosensor that detects changes within the textile or the environment surrounding the textile and responds by producing a response. The response of the biofilm may adjust, modulate or change one or more properties of the textile.

In another aspect, the present disclosure provides a biofilm-enhanced textile comprising: a textile; and a biofilm comprising one or more microorganisms and an extracellular material, the one or more microorganisms being engineered to express one or more proteins on a cell surface to adhere to the textile, and to express one or more genes that produce a response to changes within the textile or to an environment surrounding the textile.

In various embodiments, the response affects a property of the textile.

In various embodiments, the change in the environment surrounding the text is excess sweat and the one or more microorganisms is engineered to express one or more genes that produce a fragrance. For example, the one or more genes that produce the fragrance are AGPPS2 and LS.

In various embodiments, the change in the textile is mechanical stress and the one or more microorganisms is engineered to express one or more genes that produces nanofibers. For example, the one or more genes that produce nanofibers is CsgA.

In various embodiments, the one or more proteins is a bacterial nanofiber. For example, the bacterial nanofiber is a curli fiber, bacterial cellulose, or a curli fiber and bacterial cellulose. For example, the bacterial nanofiber may be engineered to bind or neutralize a virus.

In various embodiments, the one or more microorganisms is Escherichia coli, Staphylococcus epidermidis, Bacillus subtilis, Saccharomyces cerevisiae, Pichia pastoris, Pseudomonas aeruginosa, Bacteroides thetaiotaomicron, derivatives thereof, or any combination of the foregoing. For example, the one or more microorganisms is Escherichia coli or a derivative thereof.

In another aspect, the present disclosure provides methods of preparing a biofilm-enhanced textile as defined herein, the methods comprising engineering one or more microorganisms to express one or more proteins on a cell surface to adhere the cell to a textile, and to express one or more genes that produce a response to changes within the textile or to an environment surrounding the textile, and integrating the textile to a growing culture of the one or more microorganisms.

In various embodiments, the grown culture of one or more microorganisms is integrated with the textile by pouring, application with a tool, spraying or is grown directly on the textile.

In various embodiments, the grown culture of one or more microorganisms is pressed into the textile by applying mechanical pressure, pneumatic pressure, vacuum filtration or centrifugation.

In various embodiments, the methods further comprise determining a density of the biofilm on the textile.

In various embodiments, the methods further comprising stretching the textile before integrating with the growing culture of the one or more microorganisms.

In various embodiments, the methods further comprise chemically treating the textile integrated with the growing culture of the one or more microorganism to reinforce the bond between the one or more microorganisms and the textile.

In various embodiments, the methods further comprise drying, freeze-drying, starching or seeding with dried nutrient material or preservative the textile integrated with the growing culture of the one or more microorganisms.

In addition to the aspects and embodiments described above, further aspects and embodiments will become apparent by reference to the drawings and study of the following detailed description.

### BRIEF DESCRIPTION OF THE DRAWINGS

FIG. 1 shows a scheme of an example of a method of manufacturing a biofilm-enhanced textile according to an embodiment of the invention.

FIG. 2 shows microscopic images of textiles with no biofilm integrated therein as control images (upper row of images) and microscopic images of textiles with concentrated cultures of biofilm spread thereon and incubated for 24 hours (lower row of images).

FIG. 3 shows microscopic images of examples of biofilm-enhanced textiles studied under scanning electron microscopy. FIG. 3A shows biofilm integration on a textile of cotton/Lycra 94/6 formed using incubation in a concentrated biofilm solution. FIG. 3B shows biofilm integration on a textile of PET/Lycra 77/23 formed using incubation in a concentrated biofilm solution.

FIG. 4 shows microscopic images of examples of biofilm-enhanced textiles showing “self-healing” properties of the biofilm when hydrated over time.

FIG. 5 (right) shows microscopic images of a control textile with no biofilm (“control”) and three images of biofilm-enhanced textiles with various concentration of biofilm. FIG. 5 (left) is a graph showing results of the tensile tests conducted on the textiles shown in the images on the right side.

FIG. 6 shows microscopic images of examples of biofilm-enhanced textiles studied under scanning electron microscopy. FIG. 6A shows biofilm integration on a textile of cotton/Lycra 94/6 formed using paste deposition on the textile. FIG. 6B shows biofilm integration on a textile of PET/Lycra 77/23 formed using paste deposition on the textile.

FIG. 7 shows a scheme of an example for testing for biofilm expression in a textile.

FIG. 8 shows absorbance of unbound Congo Red dye for solutions from samples of cells after storage at room temperature and addition of growth media or water.

FIG. 9 shows absorbance of unbound Congo Red dye for solutions from samples of cells after lyophilization and addition of growth media or water

FIG. 10 shows absorbance of unbound Congo Red dye for solutions of concentrated biofilm of PQN4 cells and CsgA transformed cells following incubation of biofilms on textile.

FIG. 11 shows scanning electron microscopy images of a textile following cutting and rehydration with either LB growth medium (FIG. 11A) or water (FIG. 11B).

FIG. 12 shows photographs of a cut textile incorporating a biofilm as described herein (FIG. 12A) and following rehydration and growth of the biofilm to heal or repair the textile (FIG. 12B).

### DETAILED DESCRIPTION OF SPECIFIC EMBODIMENTS

In the context of the present disclosure, various terms are used in accordance with what is understood to be the ordinary meaning of those terms.

The present disclosure relates to textiles with living microorganisms that are integrated, as biofilms, into the textiles. By integrating microorganisms or microbes into the textile material, the textile can assimilate the properties of the microorganisms or microbes, such as, for example, detection and remediation of toxins, such as, for example, pathogenic bacteria or viruses. These properties can, in turn, be conferred onto the base textile-biofilm system. For example, microorganisms or microbes can have the ability to sense environmental signals and respond by generating chemical and physical changes in the textile. In addition, the biological nature of the biofilm means that it can be sourced from easily renewable raw materials and can biodegrade after disposal.

The term “biofilm” refers to a collective of one or more types of microorganisms in which cells stick to each other and to a surface. These adhered cells are embedded within an extracellular matrix that is composed of extracellular polymeric substances.

The terms “microorganism” or “microbe” refer to a microscopic organism which may exist in its single-celled form or in a colony of cells. Bacteria, fungi, viruses, protozoa and algae are the major groups of microorganisms.

The invention described herein discloses a method for generating composites of biofilms and textiles. Generation of the composite is facilitated by genetic engineering of the microbe producing the biofilm for increased adhesion to the textile material. The microbe can be further engineered for desired sense-and-respond functionality.

In one embodiment, the microbes are engineered to overexpress a secreted nanofiber, such as a curli fiber and/or bacterial cellulose, in order to increase adherence of the microbes to a textile. Nanofibers are fibers with diameters in the nanometer range. Nanofibers generally have a large surface area-to-volume ratio, high porosity and appreciable mechanical strength. Curli are amyloid proteins assembled into extracellular polymeric fibers by bacteria during biofilm formation. Curli fibers form a tough, fibrous mesh that binds individual cells together and facilitates binding to surfaces. The activation of curli gene expression can induce formation of microbe biofilms directly onto a number of materials. Gene activation can be triggered by either small molecules or light, which enables pattern formation on materials such as, for example, cotton, glass or polystyrene.

At the molecular level, curli fibers are protein polymers principally composed of the protein CsgA, which forms the primary structural component of the fibers. This protein can be genetically fused to various peptide sequences without interrupting its ability to express, polymerize, and function. These fused peptides can endow the curli fibers with novel functionalities, including the ability to nucleate amorphous crystals, bind to specific surfaces, recruit enzymes, fluoresce, or conduct charges. Therefore, by engineering the curli fibers, one can program specific chemical or physical properties into a living biofilm material.

In various embodiments, the cells within the biofilm can act as biosensors, detecting changes in the environment surrounding the textile and responding by producing one or more predictable responses such as, for example, fluorescence, a sensory response, or a change of colour. Biofilms may also produce odors in response to certain signals, such as a presence of certain toxins or excess sweat. Furthermore, the biofilm may generate enzymes that remove these toxins. These one or more responses of the biofilm may adjust, modulate or change one or more properties of the textile such as, for example, the colour of the textile, the smell of the textile, the permeability of the textile or the porosity of the textile.

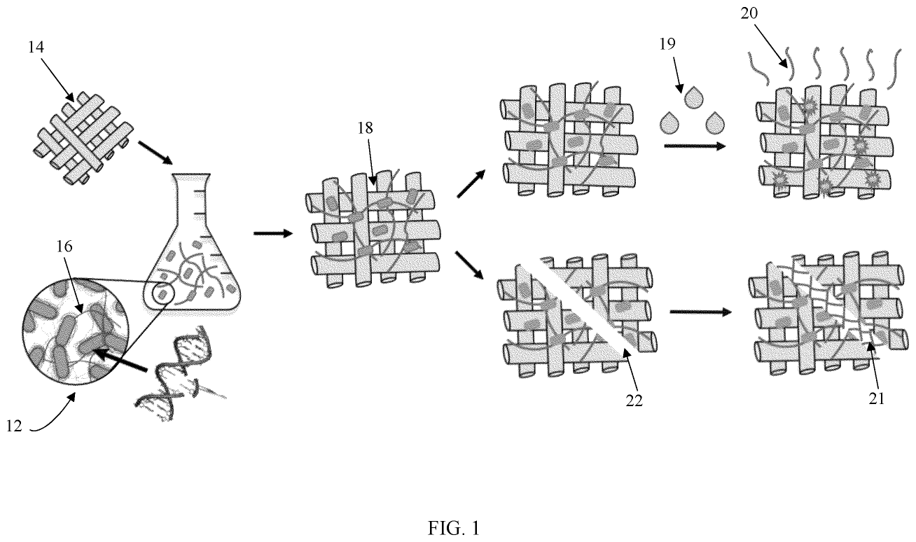

FIG. 1 illustrates a scheme of an embodiment of a method for manufacturing a biofilm-enhanced textile. Engineered microorganisms (biofilm) 12 are added to a textile 14 during culturing and biofilm integration onto the textile. The microorganisms express proteins on their surface such as, for example, curli fibers 16 that facilitate adherence to the textile 14 resulting in a biofilm/textile composite or biofilm-enhanced textile 18. Such proteins may include naturally present flagella, integrins, or adherins that may or may not have been genetically modified to increase adherence of the biofilm to the textile material as described above. For example, Escherichia coli biofilms are composed largely of curli amyloid protein, cellulose, and E. coli cells.

In some embodiments, the biofilm microorganisms integrated in the biofilm/textile composite 18 can generate fragrances 20 in response to sweat, the presence of water or increased humidity 19. For example, a PompR promoter and sensor protein from Escherichia coli can be used to generate the heterologous enzymes AGPPS2 and limonene synthase (LS), which together facilitate limonene production.

In various embodiments, the biofilm microorganisms can generate additional protein fibers 21 in response to tearing 22, resulting in self-healing of the biofilm/textile composite 18. For example, the Ppst-sigA promoter from Bacillus subtilis can be used to drive production of additional CsgA fiber protein, the production of which facilitate self-healing of the biofilm/textile composite 18.

In various embodiments, the cells of the microorganisms are integrated within and/or adhered to the textile such that the biofilm/textile composite 18 can be laundered, making the biofilm/textile composite 18 useful as a fabric for clothing.

In various embodiments, a nanofiber protein produced by the engineered microorganisms may be genetically fused to a peptide that increases the adherence of the nanofiber and cells to the textile 14. In another implementation, the nanofiber protein can be fused to a cellulose binding domain that facilitates attachment to cellulose. Cellulose is a component of various textiles, such as cotton. In yet further embodiments, the nanofiber protein can be fused to a protein domain selected by bacteriophage display for attachment to a synthetic, natural or any man-made material of interest, including, but not limited to, polyester, nylon, Lycra, cotton, bast fibers, silk, wool, lyocell, rayon, leather, polyurethane, rubber, mycelium, or a blend or composite of such synthetic, natural or man-made materials.

The microorganisms may include, but are not limited to, Escherichia coli, Staphylococcus epidermidis, Bacillus subtilis, Saccharomyces cerevisiae, Pichia pastoris, Pseudomonas aeruginosa, Bacteroides thetaiotaomicron, or common derivatives thereof as would be known to a person of ordinary skill in the art.

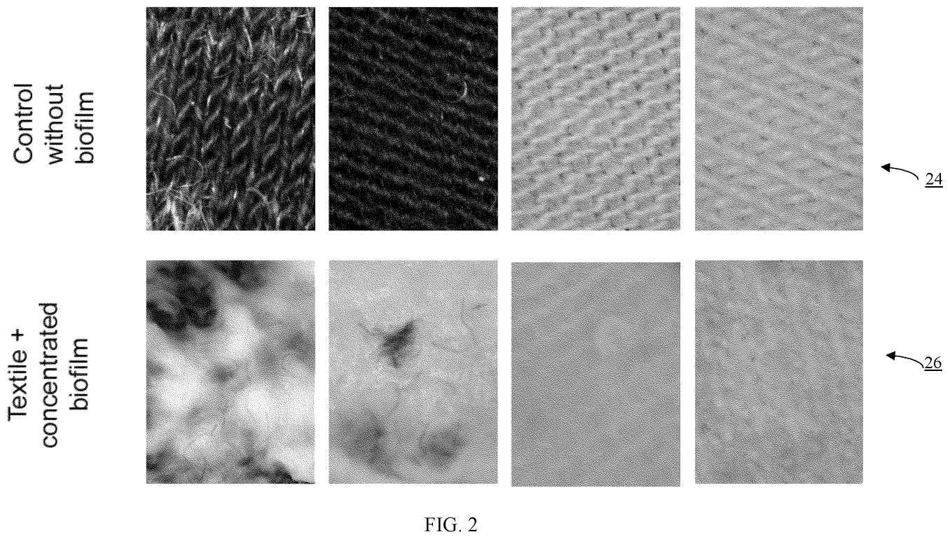

One approach for adding a biofilm onto a textile 14 is to incubate the microbes in a concentrated biofilm solution and then the concentrated solution is spread onto the textile 14. FIG. 2 illustrates microscopic images 24 of a number of textiles with no biofilm integrated therein to use them as control images. Then, concentrated cultures of biofilm-producing bacteria (microbes 12) were spread on the textiles and incubated for 24 hours. The textile/biofilm composites were then washed to remove any unintegrated biofilm from the textile and dried. Microscopic images 26 of the textiles were taken to observe the structure of such composites. Comparing images 26 with the control images 24, an opaque layer of biofilm 12 is clearly visible under the optical microscope. Thus, biofilm 12 can be integrated onto different textiles after 24 hour incubation.

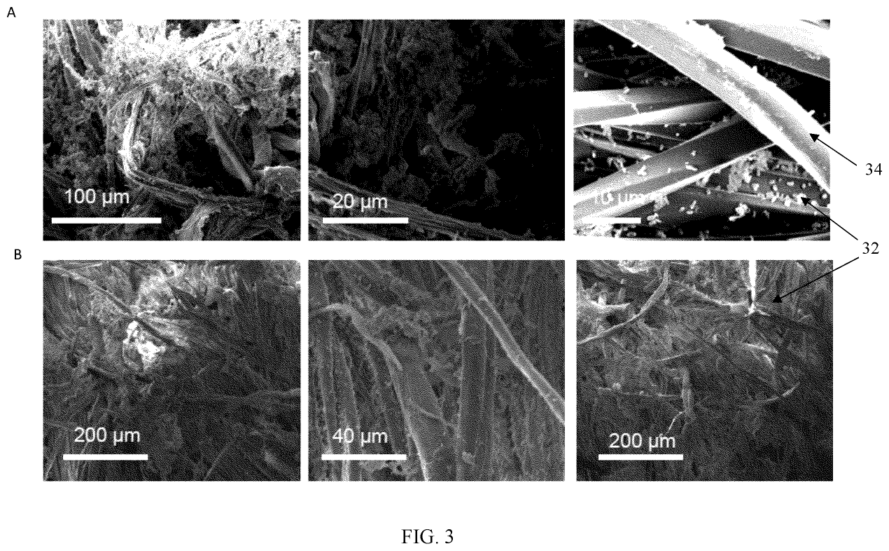

Long-term incubation with concentrated biofilms can create thick layers of curli fibers and bacterial cells (biofilms 12) throughout the textile materials 14. For example, dried biofilm/textile composites 18 were studied under scanning electron microscopy (SEM). Microscopic images of such biofilm/textile composites are shown in FIG. 3. As can be seen, biofilm material 32 is tightly integrated into textile fibers 34. FIG. 3 shows microscopic images of different textile/biofilm composites at various level of focus depth, such as 10 μm, 20 μm, 40 μm, 100 μm and 200 μm.

Direct deposition of the biofilm 12 onto the textile material 14 allowed a thicker and denser layer of curli fibers to be seen throughout the textile materials. In some embodiments, the textile material 14 can be added to a growing culture of microbes 12, or dipping or soaking textile 14 in a solution containing the growing culture of microbes 12. In one embodiment, the microbes 12 can be sprayed onto the textile 14 or can be printed or coated onto the textile 14 with pre-designed patterns. In one embodiment, the textile 14 can be stretched before being added to a growing culture of microbes 12 or before the microbes 12 are sprayed, printed or coated onto the textile 14. The stretching may improve the filling of the textile's pores with microbes 12. In another embodiment, the microbes 12 can be physically bound to the textile 14 by flowing a growing culture over sheets of the textile material in continuous flow bioreactors. In one embodiment, the microbes can be physically bound to the textile by addition of biodegradable glues or adhesives. In a further embodiment, the microbes 12 can be physically bound to the textile 14 by growing a culture of microbes on textile fibers and/or yarns and then fabricating the textile 14 with the microbe-coated textile fibers and/or yarns. In one embodiment, the microbes 12 can be physically bound to the textile 14 by spinning the microbes into textile fibers/filaments and then fabricating the textile with the microbe-incorporated textile fibers and/or yarns. In another embodiment, the microbes 12 can be physically bound to the textile 14 by incorporating them into or onto a laminate and then laminating the laminate to the textile 14. In one embodiment, the microbes 12 can be pressed into the textile 14 by applying mechanical pressure, pneumatic pressure, vacuum filtration, or by centrifugation force.

After the microbes 12 are integrated with the textile 14, it can be chemically treated to reinforce the bond between the microbes 12 and the textile 14. In one embodiment, the textile/microbe composite can then be dried or freeze-dried as a preservation step. In another embodiment, the composite can be treated with starch or seeded with dried nutrient material to provide nutrient support for later biofilm growth,

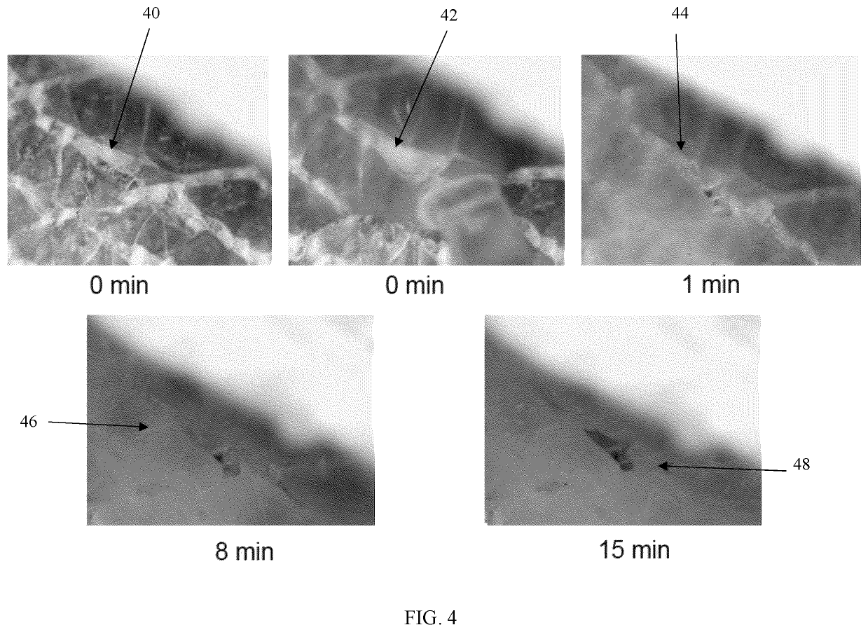

Images shown in FIG. 4 demonstrate “self-healing” properties of the biofilm-enhanced textiles. In various embodiments, a high concentration of biofilm-producing bacteria was used to form a biofilm on a textile for 24 hours and the biofilm/textile composite was subsequently dried. Then, physical cracks were induced in the biofilm/textile composite with mechanical shearing (see image 40). A drop of water was then applied to the biofilm/textile composite (see image 42 showing the biofilm immediately after the water is added at time 0 min) to induce hydration of the composite. Image 44 shows the reduction of the size of the physical cracks just after 1 min hydration time with further reduction in the number and size of physical cracks in the biofilm/textile composite shown in images 46 and 48 after 8 min and 15 min hydration time, respectively.

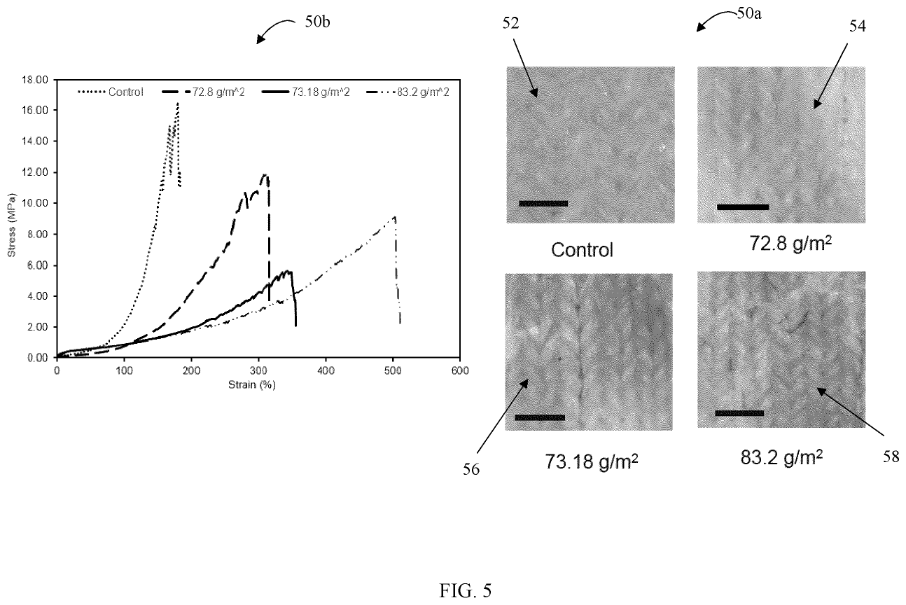

The characteristics of the obtained biofilm-enhanced textile composites 18 were also tested to evaluate, for example, material ductility during tensile tests. Ductility as defined herein means the ability of a material to stretch before breaking/rapture. For example, each end of a piece of the biofilm-enhanced textile composite 18 was clamped and then stretched, increasing the stress applied to the composite 18. Tensile tests to control textiles with no biofilms were also conducted for comparison. The tensile tests were conducted in the direction of the warp and weft of each textile. FIG. 5 shows the results of some of the conducted tensile tests. Images 50a on the right side are images of the tested textiles. Image 52 is the control textile with no biofilm thereon, whereas image 54 shows textile integrated with 72.8 g/m² biofilm, image 56 shows textile integrated with 72.8 g/m² biofilm and image 58 shows textile integrated with 83.2 g/m² biofilm. Diagram 50b on the left side shows the results of the tensile tests. As can be seen, concentrated biofilms on textile improves its ductility. For example, curve 51 illustrates the results of the tensile tests of the control textile 52 showing that the control textile breaks at the lowest percentage of strain force while the highest concentration of biofilm on the textile (curve 57) breaks at highest strain force.

In various embodiments, the microbes 12 can be induced to adhere to the textile 14 by chemical induction. For example, the microbes can be induced to adhere to the textile by light, by changes in temperature, by sonication, by application of electric current, or any other method as would be known to a person of ordinary skill in the art.

Genetic modifications made to the microbes 12 may be encoded on plasmids or on the genome of the microbe. For example, modifications may include elements such as resistance markers, chemical dependencies (auxotrophies), and origins of replication. In one embodiment, functional microbes such as nitrifying bacteria, cyanobacteria, actinobacteria or algae, can be integrated in the textile/biofilm composite 18 together with the adhering microbes for added functionality. In one embodiment, functional organic or inorganic ingredients such as sunlight blocking agents, antibacterial nanoparticles, cyclodextrin, medical ingredients or skin care agents can be integrated in the textile/biofilm composite 18 together with the adhering microbes for added functionality.

The functions and/or features that can be facilitated by the biofilm-enhanced textiles may include, but are not limited to:
a) Responsive scenting: by sensing sweat, the microbes feed on sweat and generate a scent.
b) Responsive water and vapor guiding/channeling: by sensing the micro and ambient environment, the microbes alter the biofilm structure/pattern in order to channel sweat and vapor from the microenvironment to the exterior of the textile; or prevent water penetration with increased breathability with rain channeling structures.
c) Responsive modulus-strain alteration: by sensing the strain and the vibration frequency, the microbes alter the modulus and the stretch of the biofilm to make the textile more/less resistant to further perturbation.
d) Adaptive venting: when the humidity in the microenvironment increases, the microbes will respond causing the biofilm to change shape, enlarging the textile pore structures to allow for active venting.
e) Self-cleaning textiles: the microbes produce an amphiphobic (hydrophobic and oleophobic) biofilm that repels water-based and oil-based stains, and reduces the need to clean and launder a textile.
f) Anti-viral: the microbes can either strongly bind or neutralize a particular virus in such a way as to prevent its transmission to the wearer.
g) Anti-microbial: the microbes can be engineered to kill pathogenic germs, such as, for example, pathogenic bacteria.
h) Skin microbiome: the microbes produce a biofilm that complements the skin microbiome of the wearer. For example, producing an environment that reduces the growth of bacteria associated with body odour on the skin and in textiles, or fostering the growth of commensal skin microorganisms.
i) Air purification: the microbes in the textile can kill pathogenic bacteria and/or viruses, degrade volatile chemicals and collect floating particles such as, for example, microplastics, which can be released by washing or encountered in the ambient environment.
j) Responsive to sweat: the microbes sense specific metabolites found in human sweat and use that input to promote further biofilm formation and/or to alter biofilm properties. For example, the thermal insulation provided by the biofilm(s) may be altered.
k) Responsive release: the microbes and the biofilm sense temperature or humidity change, and in response, slowly release one or more compounds such as, for example, skin nutritive ingredients, scent agents, sedative agents, menthol or water.
l) Responsive to friction and/or drag in air and/or water: the microbes alter the biofilm surface in response to friction and drag, depending on the purpose. For example, smoothing a biofilm surface to reduce friction and reduce blister risk in key locations based on increases in detected friction, or increasing biofilm surface friction to increase tactile information in glove fingertips.
m) Adaptive thermal insulation: the microbes generate a more porous and thicker biofilm in response to the repeated detection (such as, for example, multiple consecutive days) of a low temperature below a predefined threshold, and breakdown the biofilm to create a thinner, less insulative, biofilm in response to the repeated detection of a higher temperature above a predefined threshold.
n) Responsive luminescence or colouration: the microbes produce a biofilm that luminesces or changes colours or dye production in response to a sensed input such as, for example, metabolites, the presence of sunlight or the absence of sunlight.
o) Base material: the biofilm is used as a base material for the incorporation of functional materials or symbiotic living microorganisms such as, for example, nanoparticles, cyclodextrin, graphene, probiotics, algae, lichen or fungi.

The functional characteristics of biofilms have been investigated by several research groups. For example, Horvat et al, “Viscoelastic response of E. coli biofilms to genetically altered expression of extracellular matrix components”, Soft Matter 15, 5042-5051 (2019), disclose engineered multiple strains of Escherichia coli with varying composition of its biofilm's extracellular matrix. These modifications revealed changes in the viscoelastic behavior of the biofilm, demonstrating that the biofilm composition can be genetically programmed to achieve specific mechanical properties.

The versatility of biofilms has also been exploited to expand the properties of 3D-printed polymers. Huang et al, “Programmable and printable Bacillus subtilis biofilms as engineered living materials”, Nat. Chem. Biol. 15, 34-41 (2019) teach the integration of Bacillus subtilis biofilms into layers of hydrogels to create a wide array of three-dimensional shapes and microencapsulated structures containing living, responsive cells.

In another example, Raab, et al., “A symbiotic-like biologically-driven regenerating fabric”, Sci Rep 7, 8528, doi: 10.1038/s41598-017-09105-4 (2017), demonstrated that Bacillus subtilis engineered to produce spider silk protein could be adhered to textile and facilitate the formation of spider silk nanofibers upon lysis of the cells due to mechanical shear.

Additionally, Nguyen et al., “Programmable biofilm-enhanced materials from engineered curli nanofibers”, Nat. Commun. 5, 1-10 (2014); Dorval et al., “Biomimetic engineering of conductive protein films”, Nanotechnology 29(45), 454002 (2018); Barnhart et al., “Curli biogenesis and function”, Annu. Rev. Microbiol. 60, 131-47 (2006); Moser et al., “Light-Controlled, High-Resolution Patterning of Living Engineered Bacteria Onto Textiles”, Ceramics, and Plastic, Adv. Funct. Mater. 29, 1901788 (2019) and Dorval et al., “Scalable Production of Genetically Engineered Nanofibrous Macroscopic Materials via Filtration”, ACS Biomater. Sci. Eng. 3, 733-741 (2017), demonstrate how engineered biofilms can endow diverse inorganic materials with properties typically confined to living systems.

### EXAMPLES

These examples illustrate various aspects of the invention, evidencing a variety of conditions for preparing biofilm-enhanced textiles and methods of making same. Selected examples are illustrative of advantages that may be obtained compared to alternative textiles, and these advantages are accordingly illustrative of particular embodiments and not necessarily indicative of the characteristics of all aspects of the invention.

### Example 1: Low Density Biofilm Integration on Textiles

Integration methods that may result in a lower density biofilm on the textile include incubating a textile in a biofilm solution and depositing a paste of biofilm on the textile.

For incubation of the textile in the concentrated biofilm solution, a bacterial culture of E. coli was expressed overnight to an optical density of about 2.7. The culture was centrifuged and a growth medium, such as LB medium, was added to reach a concentration of about 0.4 g/mL. The textile was then added and the biofilm solution was incubated for 24 hours at room temperature. The textile was fixed and studied under a scanning electron microscope. FIG. 3A shows biofilm integration on a textile of cotton/Lycra 94/6 at a scale of 100 μm, 20 μm and 10 μm. FIG. 3B shows biofilm integration on a textile of PET/Lycra 77/23 at a scale of 200 μm, 40 μm and 200 μm. As can be seen from FIGS. 3A and 3B, incubation of the textile resulted in widespread distribution of cells and curli fibers throughout the matrix.

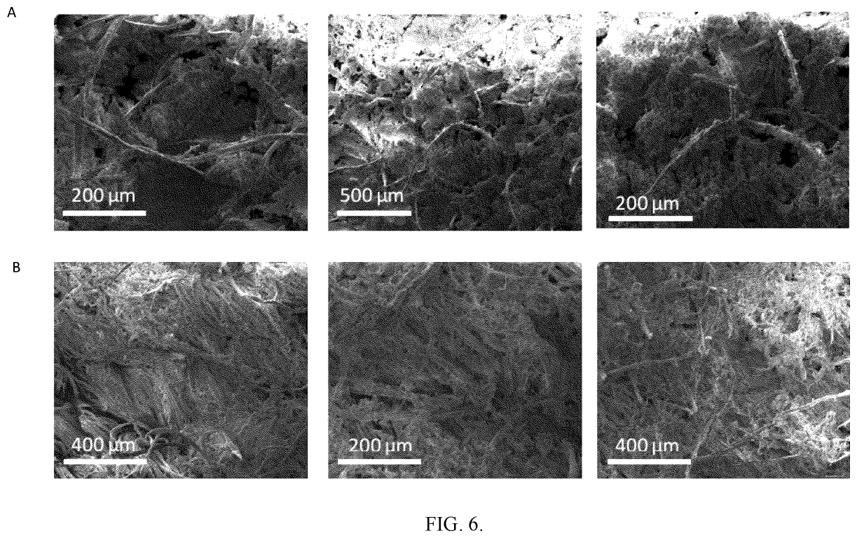

For paste deposition of a biofilm on a textile, a bacterial culture of E. coli was expressed overnight to an optical density of about 2.7. The culture was centrifuged and a growth medium, such as LB medium, was added to reach a concentration of approximately 2 g/mL. Following a period of time to allow biofilm formation, the paste was spread over a pre-wet textile with a spatula. FIG. 6A shows biofilm integration on a textile of cotton/Lycra 94/6 at a scale of 200 μm, 500 μm and 200 μm. FIG. 6B shows biofilm integration on a textile of PET/Lycra 77/23 at a scale of 400 μm, 200 μm and 400 μm. As can be seen from FIGS. 6A and 6B, paste deposition of the biofilm on the textile resulted in a thick coating of cells and biofilm on the textile surface.

In various embodiments, these lower density integration protocols may be preferred for applications that do not require changes in the mechanical properties of the textile or only require changes in surface properties. For example, for applications relating to the release of fragrance molecules from the textile, the lower density integration protocols may be used. As described below, using a lower density protocol, about 100 to about 450 mg of limonene/L of culture deposited on the textile could be produced.

### Example 2: High Density Biofilm Integration on Textiles

Integration methods that may result in a higher density biofilm on the textile include vacuum filtration of the biofilm on the textile. For example, a bacterial culture of E. coli was expressed overnight to an optical density of about 2.7. The culture was centrifuged and a growth medium, such as LB medium, was added to reach a concentration of about 0.50 g/mL. The textile was fixed on the vacuum filter and in some embodiments, was also stretched. The stretching may improve pore-filling of the textile. The biofilm solution was added while the vacuum was on, until the filtration slowed to approximately 3-5 mL through the textile with a diameter of 3 cm. The textile was dried overnight.

This protocol resulted in dense biocomposites on the textile that had an ability to self-heal with water, as described below. Higher density protocols may be useful in applications that require changes in mechanical properties of the textile, for uniform functionalization throughout the textile matrix, and/or to introduce self-healing properties onto the integrated textile.

### Example 3: Combination of Biofilm Filtration and Incubation

A protocol using a combination of biofilm filtration and incubation may result in a more uniform distribution of biofilm in the textile and improved self-healing properties in water. The textile was pre-incubated in a bacterial culture for about 30-60 minutes as described in Example 1 and then placed over a vacuum filter. One or more additions of a biofilm solution having a concentration of about 2.5 g/mL was added. The vacuum was left off for about 30 minutes and turned on for about 1 minute or left on continuously until the filtration slowed. The textile was then dried overnight at room temperature.

### Example 4: Biofilm-Enhanced Textile Responsive to Sweat

In one non-limiting embodiment, microbes were engineered to respond to sweat. A sweat-sensitive promoter, such as for example, PompR from Escherichia coli, was genetically fused to a gene or series of genes that encode a specified output which may include, but is not limited to, the genes AGPPS2 and LS that comprise the limonene synthase pathway. Persons skilled in the art understand that other known pathways that generate fragrances can be used without departing from the scope of the invention. Then the genetically modified PompR from Escherichia coli was bound to a textile as described above. The genetic modifications to the PompR promoter enable the microbe to sense sweat and respond by generating a fragrance. For example, E. coli cells bearing plasmid pJBEI6410 were cultured to an optical density of 1. Protein expression was induced with 25-500 μM IPTG and the biofilm was grown for about 24-120 hours, during which time limonene was produced by the E. coli cells. A layer of n-dodecane was added on top of the biofilm solution following addition of the IPTG. The limonene was trapped in the organic layer and quantified with FC-FID.

### Example 5: Biofilm-Enhanced Textile Responsive to Environmental or Mechanical Stress

In another non-limiting embodiment, the microbes were genetically engineered to respond to mechanical stresses. In this embodiment, a shear-sensitive promoter such as, but not limited to, Ppst-sigA from Bacillus subtilis, was genetically fused to a gene or series of genes that encode a specific output. The specific output may include, but is not limited to, CsgA or other genes encoding nanofiber formation. Increasing nanofiber formation may increase and strengthen the extracellular matrix of the biofilm, thereby stiffening the composite.

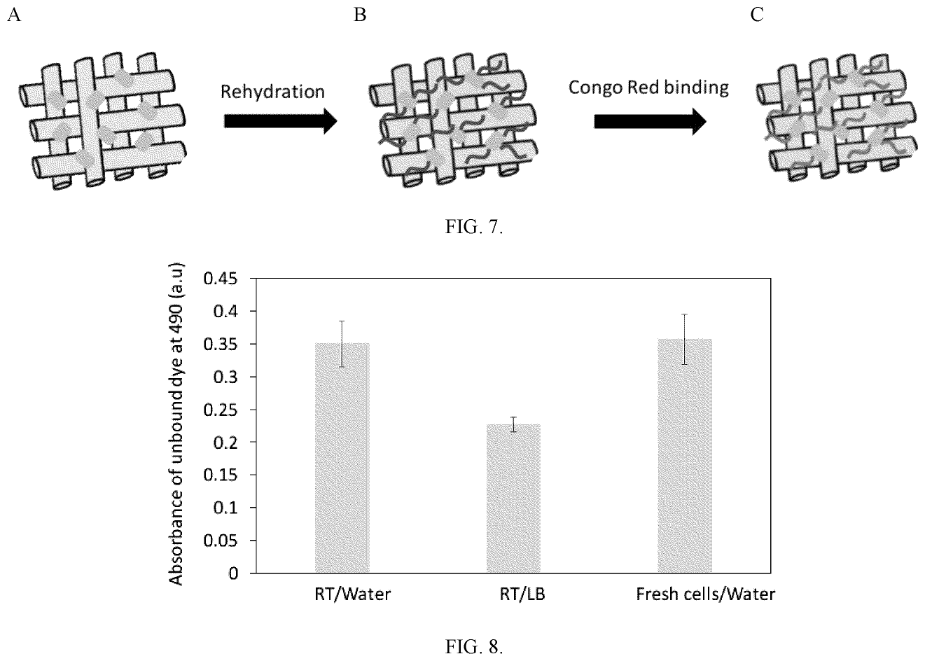

As shown in FIG. 7, bacterial cells may be integrated on textiles at the log phase of bacterial growth and then dried at room temperature (A). Following rehydration, the cells may produce curli fibers (B). Congo Red may be added to bind to the curli fibers for visual detection of biofilms (C).

In various embodiments, it was shown that the cells can express biofilm after storage at room temperature without fresh nutrients. A sample (1 mL) of 0.55 optical density cell culture was stored at room temperature overnight. Fresh growth media, such as LB media, or water (1 mL) was added and the biofilm was incubated overnight at 37° C. Expression of curli fibers was tested using the Congo Red assay (“RT/LB” in FIG. 8). As shown in FIG. 8, curli fibers were detected even for samples where only water at room temperature was added to the cell culture (“RT/Water”) or only water was added to fresh cells (“Fresh cells/Water”).

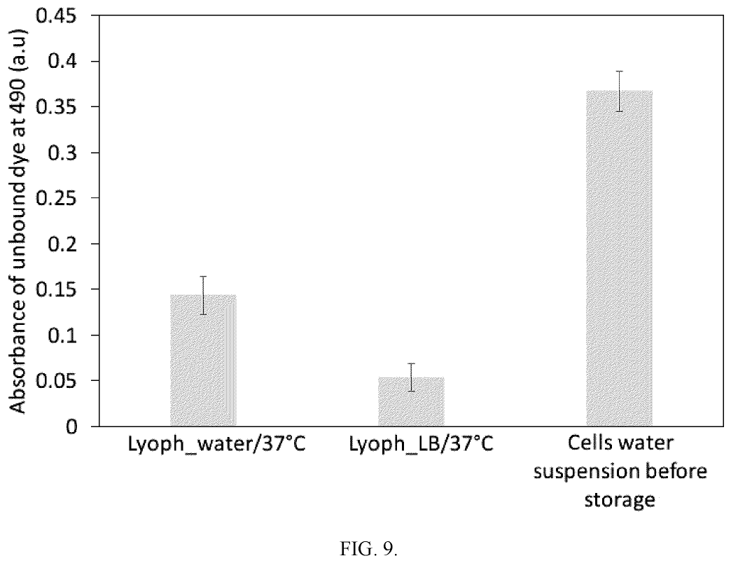

The cells were also tested to determine whether biofilm could be expressed after a cycle of freeze/drying. A 1 mL sample of 0.55 optical density cell culture was lyophilized by being held for 12 hours at −20° C., followed by 12 hours of deep vacuum. Either fresh LB media or water (1 mL) was added to the cell culture and incubated overnight at 37° C. The extent of biofilm formation was tested using the Congo Red assay. As shown in FIG. 9, the cells were able to express biofilm even after lyophilization, regardless of whether water was added (“Lyoph_water/37° C.” in FIG. 9) or growth media was added (“Lyoph_LB/37° C.” in FIG. 9) to the cultured cells. As a control, these results were compared to absorbance of unbound dye in a sample of cells in water before storage (“Cells water suspension before storage” in FIG. 9), indicating the formation of biofilm.

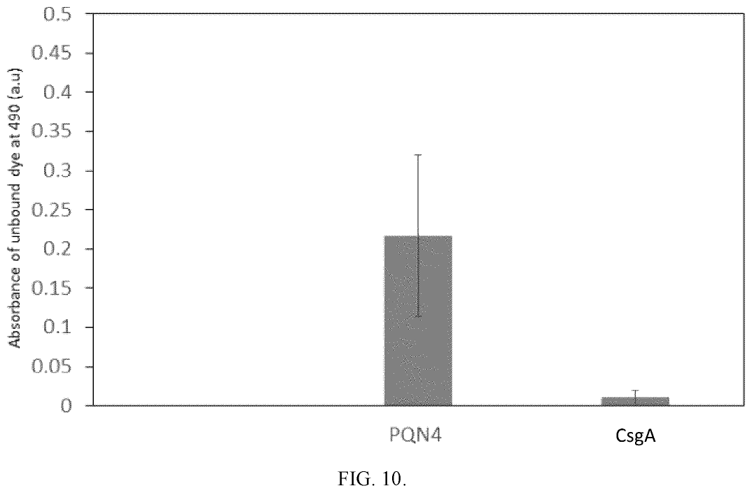

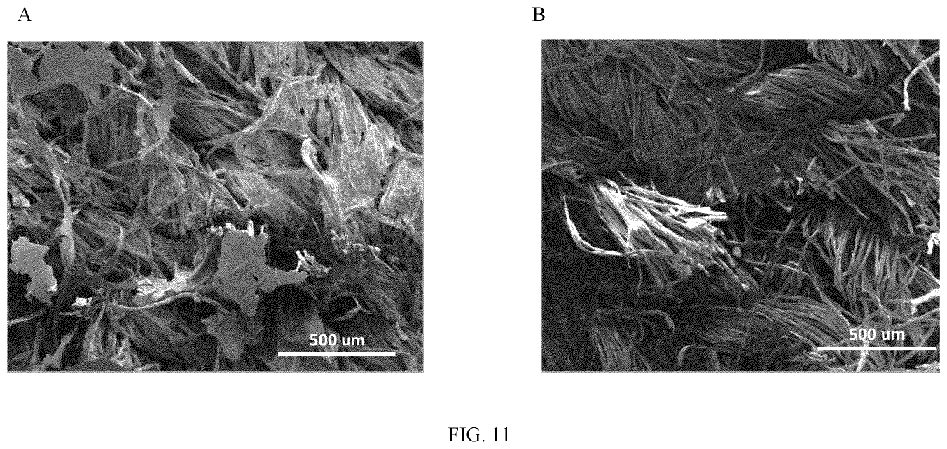

It was then determined whether integration of the cells on textile would interfere with biofilm formation. Cells were integrated on textile as described in Examples 1-3 above. Cells were resuspended in water (at a concentration of 0.2 g/mL) and integrated with textiles using 1 mL of the suspension for 1 cm² of textile. Two different cell types were tested, PQN4 cells as a control and the CsgA transformed cells as described above. PQN4 is an E. coli derived strain of cells that lacks the entire curli operon. The textile was added and incubated overnight in 2 mL of LB media at 37° C. A 1 mL sample of each solution was taken for Congo Red binding assay. The results are shown in FIG. 10. As expected, the PQN4 alone cells did not express curli fibers and therefore, the Congo Red dye was free in solution, resulting in a higher absorbance in the assay. In contrast, the CsgA transformed cells still expressed curli fibers to which the Congo Red dye was bound, resulting in a reduced absorbance of unbound dye in the assay. These results indicate that integration of the cells on textile did not affect their ability to express biofilm. The textiles were then dried and tested to determine whether the cells integrated on textile could express biofilm after drying. Following drying of the textiles for 1 day, 3 days or 4 days, for textiles incorporating either the PQN4 cells or the CsgA transformed cells, the cells could still express biofilm following placement back in a growth media.

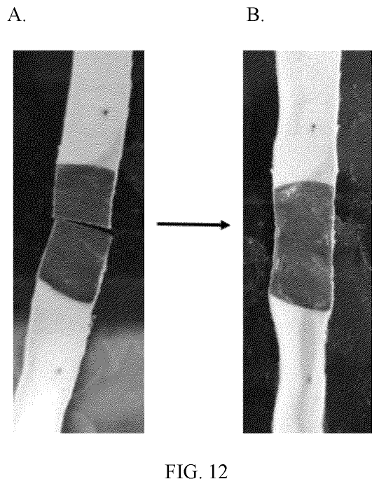

Textiles prepared according to Examples 1, 2 and 3 as described above were cut with scissors. About 100-200 μL of water was added to the cut area and the text was left to heal (or dry) for about 30-60 minutes. Two different conditions were tested, either the textile was rehydrated with LB growth media and incubated overnight at room temperature, or the textile was rehydrated with water and incubated overnight at room temperature. Scanning electron microscopy showed that for both rehydration conditions, rehydration with LB growth media in FIG. 11A and rehydration with water in FIG. 11B, expression of the biofilm was able to partially fill the cut area. FIG. 12 shows photographs of the cut textile (FIG. 12A) and the healed textile (FIG. 12B), demonstrating the development of the biofilm through generation of curli fibers to reconnect the pieces of textile. Thus, biofilms produced from the CsgA transformed cells may be used for textile self-repair.

While particular elements, embodiments and applications of the present disclosure have been shown and described, it will be understood that the scope of the disclosure is not limited thereto, since modifications can be made by those skilled in the art without departing from the scope of the present disclosure, particularly in light of the foregoing teachings. Thus, for example, in any method or process disclosed herein, the acts or operations making up the method/process may be performed in any suitable sequence and are not necessarily limited to any particular disclosed sequence. Elements and components can be configured or arranged differently, combined, and/or eliminated in various embodiments. The various features and processes described above may be used independently of one another, or may be combined in various ways. All possible combinations and subcombinations are intended to fall within the scope of this disclosure. Reference throughout this disclosure to “some embodiments,” “an embodiment,” or the like, means that a particular feature, structure, step, process, or characteristic described in connection with the embodiment is included in at least one embodiment. Thus, appearances of the phrases “in some embodiments,” “in an embodiment,” or the like, throughout this disclosure are not necessarily all referring to the same embodiment and may refer to one or more of the same or different embodiments. Indeed, the novel composites, textiles and methods described herein may be embodied in a variety of other forms; furthermore, various omissions, additions, substitutions, equivalents, rearrangements, and changes in the form of the embodiments described herein may be made without departing from the disclosure described herein.

Various aspects and advantages of the embodiments have been described where appropriate. It is to be understood that not necessarily all such aspects or advantages may be achieved in accordance with any particular embodiment. Thus, for example, it should be recognized that the various embodiments may be carried out in a manner that achieves or optimizes one advantage or group of advantages as taught herein without necessarily achieving other aspects or advantages as may be taught or suggested herein.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without operator input or prompting, whether these features, elements and/or steps are included or are to be performed in any particular embodiment. No single feature or group of features is required for or indispensable to any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

The example calculations, simulations, results, graphs, values, and parameters of the embodiments described herein are intended to illustrate and not to limit the disclosed embodiments. Other embodiments can be configured and/or operated differently than the illustrative examples described herein

1. A biofilm enhanced textile comprising:
a textile; and
a biofilm comprising one or more microorganisms and an extracellular material, the one or more microorganisms being engineered to express one or more proteins on a cell surface to adhere to the textile, and to express one or more genes that produce a response to changes within the textile or to an environment surrounding the textile.

2. The biofilm of claim 1, wherein the response affects a property of the textile.

3. The biofilm of claim 1, wherein the change in the environment surrounding the textile is excess sweat and the one or more microorganisms is engineered to express one or more genes that produce a fragrance.

4. The biofilm of claim 3, wherein the one or more genes that produce the fragrance are AGPPS2 and LS.

5. The biofilm of claim 1, wherein the change within the textile is mechanical stress and the one or more microorganisms is engineered to express one or more genes that produces nanofibers.

6. The biofilm of claim 5, wherein the one or more genes that produce nanofibers is CsgA.

7. The biofilm of claim 1, wherein the one or more proteins is a bacterial nanofiber.

8. The biofilm of claim 7, wherein the bacterial nanofiber is a curli fiber, bacterial cellulose, or a curli fiber and bacterial cellulose.

9. The biofilm of claim 7, wherein the bacterial nanofiber is engineered to bind or neutralize a virus.

10. The biofilm of claim 1, wherein the one or more microorganisms is Escherichia coli, Staphylococcus epidermidis, Bacillus subtilis, Saccharomyces cerevisiae, Pichia pastoris, Pseudomonas aeruginosa, Bacteroides thetaiotaomicron, common derivatives thereof, or any combination of the foregoing.

11. The biofilm of claim 10, wherein the one or more microorganisms is Escherichia coli or a common derivative thereof.

12. A method of preparing a biofilm-enhanced textile as defined in claim 1, comprising:
engineering one or more microorganisms to express one or more proteins on a cell surface to adhere the cell to a textile, and to express one or more genes that produce a response to changes within the textile or to an environment surrounding the textile; and
integrating the textile to a growing culture of the one or more microorganisms.

13. The method of claim 12, wherein the grown culture of one or more microorganisms is integrated with the textile by pouring, application with a tool, spraying or is grown directly on the textile.

14. The method of claim 12, wherein the grown culture of one or more microorganisms is pressed into the textile by applying mechanical pressure, pneumatic pressure, vacuum filtration or centrifugation.

15. The method of claim 12, further comprising determining a density of the biofilm on the textile.

16. The method of claim 12, further comprising stretching the textile before integrating with the growing culture of the one or more microorganisms.

17. The method of claim 12, further comprising chemically treating the textile integrated with the growing culture of the one or more microorganism to reinforce the bond between the one or more microorganisms and the textile.

18. The method of claim 12, further comprising drying, freeze-drying, starching or seeding with dried nutrient material or preservative the textile integrated with the growing culture of the one or more microorganisms.
